SEQUENCE LISTING (14 sequences)

SEQ ID NO: 1 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
GFSFSSYG

SEQ ID NO: 6 - protein, 8 aa, synthetic construct
ISPSGSYF

SEQ ID NO: 7 - protein, 12 aa, synthetic construct
ARLPGFNPPFDH

SEQ ID NO: 8 - protein, 6 aa, synthetic construct
QNVGGN

SEQ ID NO: 9 - skipped

SEQ ID NO: 10 - protein, 9 aa, synthetic construct
LQYKNNPWT

SEQ ID NO: 11 - protein, 448 aa, synthetic construct
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

(19) United States
(12) Patent Application Publication
(10) Pub. No.: US 2026/0015416 A1
(43) Pub. Date: Jan. 15, 2026

(54) TREATMENT OF DRUG-RESISTANT HEPATOCELLULAR CARCINOMA

(71) Applicants: Alentis Therapeutics AG, Basel (CH); Université de Strasbourg, Strasbourg (FR); Institut National de la Santé et de la Recherche Médicale, Paris (FR); Hôpitaux Universitaires de Strasbourg, Strasbourg (FR)

(72) Inventors: Thomas Fredy BAUMERT, Strasbourg (FR); Markus KJ MEYER, Allschwil (CH); Roberto IACONE, Allschwil (CH); Alberto TOSO, Allschwil (CH); Hong Tuan DUONG, Fortschwihr (FR)

(21) Appl. No.: 19/116,677

(22) PCT Filed: Oct. 2, 2023
(86) PCT No.: PCT/EP2023/077233

§ 371 Date: Mar. 28, 2025

Related U.S. Application Data
(60) Provisional application No. 63/377,861, filed on Sep. 30, 2022.

(51) Int. Cl. C07K 16/28; A61K 39/00; A61P 35/00

(52) U.S. Cl. CPC C07K 16/28; A61P 35/00; C07K 16/2818; A61K 2039/507; C07K 2317/24; C07K 2317/565

(57) ABSTRACT

The present disclosure relates to a method of treating hepatocellular carcinoma in a human subject comprising administering an anti-Claudin-1 antibody to a human subject, wherein the hepatocellular carcinoma is resistant to sorafenib and/or PD-1 antagonists.

### CROSS REFERENCE TO RELATED APPLICATIONS

This application claims the benefit of and the priority to U.S. Provisional Patent Application No. 63/377,861, filed Sep. 30, 2022, the contents of which are incorporated by reference herein in their entirety.

### REFERENCE TO AN ELECTRONIC SEQUENCE LISTING

The contents of the electronic sequence listing (ALNT-011_001WO_SeqList_ST26.xml; Size 14,194 bytes; and Date of Creation: Sep. 28, 2023) are herein incorporated by reference in their entireties.

### FIELD

The present disclosure relates to methods for treating sorafenib-and/or nivolumab-resistant hepatocellular carcinoma.

### BACKGROUND OF THE INVENTION

Hepatocellular carcinoma (HCC) is a major public health burden and the fourth leading and fast rising cause of cancer-related death worldwide (Llovet J M, et al., Nat Rev Dis Primers 2021; 7:6). HCC typically develops on the background of advanced liver fibrosis caused by viral or metabolic injury. Irrespective of the etiology, hyperactivation of oncogenic signaling such as the Ras/Raf/MAPK, PI3K/AKT/-mTOR, Notch and Wnt/B-catenin pathways are common events involved in HCC initiation and progression. Moreover, the tumor microenvironment (TME) plays a key role for HCC outcome (Llovet et al.).

Current treatment options for advanced HCC are still unsatisfactory due to limited response rates (Llovet et al., and Finn R S, et al., N Engl J Med 2020; 382:1894-905). Therapeutic resistance to current systemic therapies has been associated with tumor cell plasticity, such as epithelial-mesenchymal (EMT) transition and stemness, as well as an immune-exhausted or immune-excluded TME (Llovet et al.; Calderaro J, et al., J Hepatol 2019; 71:616-30; and Qin S, et al., Signal Transduct Target Ther 2020; 5:228). Persistence of pro-tumorigenic signals within the fibrotic niche on the other hand accounts for high risk of tumor recurrence following curative treatment approaches (Llovet et al.). There is a need for novel HCC therapeutics that address the drawbacks of drug resistance and tumor recurrence. Provided herein are methods and compositions addressing this need.

### SUMMARY OF THE INVENTION

Provided herein is a method for identifying a human subject having hepatocellular carcinoma (HCC) that is suitable for therapy with an anti-Claudin-1 antibody or antigen binding fragment thereof comprising the steps of: a) obtaining a biological sample a human subject having HCC; b) detecting the expression of Claudin-1; c) comparing the detected expression level of Claudin-1 with a control level of expression; and d) identifying the human subject as a responder when the detected expression level of Claudin-1 is greater than the control level of expression. In some embodiments, the method comprises: e) administering an anti-Claudin-1 antibody or antigen binding fragment thereof in an amount sufficient to alleviate a symptom of HCC when the human subject is identified as a responder.

Provided herein is a method for treating a human subject having HCC comprising the steps of: a) obtaining a biological sample from a human subject having HCC; b) detecting the expression levels of Claudin-1; c) comparing the detected expression level of Claudin-1 with a control level of expression; d) identifying the human subject as a responder when the detected expression level of Claudin-1 is greater than the control level of expression; and e) administering an anti-Claudin-1 antibody or antigen binding fragment thereof in an amount sufficient to alleviate a symptom of HCC when the human subject is identified as a responder.

In some embodiments, the control level of expression is determined from a normal tissue sample, and wherein the normal tissue sample is adjacent to the biological sample from the human subject having HCC.

Provided herein is a method of treating HCC in a human subject, comprising administering a therapeutically effective amount of an anti-Claudin-1 antibody or an antigen binding fragment thereof. In some embodiments, the HCC is resistant to sorafenib, a PD-1 antagonist and/or a PD-L1 antagonist. In some embodiments, the HCC is resistant to sorafenib. In some embodiments, the HCC is resistant to a PD-1 antagonist. In some embodiments, the HCC is resistant to PD-L1 antagonist.

In some embodiments, the anti-Claudin-1 antibody or antigen binding fragment thereof comprises: a CDRH1 comprising the amino acid sequence of SEQ ID NO: 5; a CDRH2 comprising the amino acid sequence of SEQ ID NO: 6; a CDRH3 comprising the amino acid sequence of SEQ ID NO: 7; a CDRL1 comprising the amino acid sequence of SEQ ID NO: 8; a CDRL2 comprising the amino acid sequence of GA; and a CDRL3 comprising the amino acid sequence of SEQ ID NO: 10.

In some embodiments, the anti-Claudin-1 antibody or antigen binding fragment thereof comprises a heavy chain variable region comprising an amino acid sequence of SEQ ID NO: 3 and a light chain variable region comprising an amino acid sequence of SEQ ID NO: 4.

In some embodiments, the anti-Claudin-1 antibody or antigen binding fragment thereof comprises a heavy chain variable region comprising an amino acid sequence of SEQ ID NO: 13 and a light chain variable region comprising an amino acid sequence of SEQ ID NO: 14.

In some embodiments, the anti-Claudin-1 antibody is a humanized antibody.

In some embodiments, the HCC is resistant to sorafenib or a PD-1 antagonist.

In some embodiments, the human subject was previously treated with sorafenib. In some embodiments, the human subject is simultaneously or sequentially administered with a therapeutically effective amount of sorafenib.

In some embodiments, the human subject was previously treated with a PD-1 antagonist. In some embodiments, the human subject is simultaneously or sequentially administered with a therapeutically effective amount of PD-1 antagonist. In some embodiments, the PD-1 antagonist is nivolumab, pembrolizumab, cemiplimab, dostarlimab or a combination thereof. In some embodiments, the PD-1 antagonist is nivolumab.

In some embodiments, the human subject was previously treated with a PD-L1 antagonist. In some embodiments, the human subject is simultaneously or sequentially administered with a therapeutically effective amount of PD-L1 antagonist. In some embodiments, the PD-1 antagonist is atezolizumab (TECENTRIQ; RG7446), or durvalumab (IMFINZI; MEDI4736) or avelumab (Bavencio) or a combination thereof.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A, 1B and 1C show that anti-Claudin-1 monoclonal antibodies (“H3L3” antibody) inhibit viability in patient-derived ex vivo models of HCC. FIG. 1A shows representative microscopic photos of tumor spheroids generated from HCC liver tissue treated with a anti-Claudin-1 monoclonal antibody (“H3L3”) or control mAb on day 6 post-treatment are shown. Scale bars 200 μm. FIG. 1B depicts graphs showing relative cell viability after 6 days of treatment with anti-Claudin-1 mAb “H3L3” (Table 1) or sorafenib compared to control mAb treated spheroids (n=3 replicates per condition, p=0.003 and p=0.04, Student's t-test). Bars show mean±SEM. *p<0.05, **p<0.01, ***p<0.001, **** p<0.0001. FIG. 1C depicts a heatmap showing cell viability of tumor spheroids (n=15 donors) were treated with anti-Claudin-1 mAb “H3L3”, control Ab, sorafenib or nivolumab. Heatmaps illustrate % cell viability compared to control mAb treated cells on day 6 using ATP quantification (n=15 different donors with at least duplicates per condition). Donors (# ST1, # S06, # S15, # S16, # S17, # S18, # R2, # R21, # R24, # R11, # 381, # S19, # S22, # S349, # S394).

### DETAILED DESCRIPTION

In a large number of hepatocellular carcinoma (HCC) subjects, the effectiveness of conventional therapies are hampered by the development of drug resistance. Since HCC is highly heterogeneous, within the tumor and among individuals, this influences disease progression, classification, prognosis, and naturally cellular susceptibility to drug resistance. Further, persistence of pro-tumorigenic signals in the tumor microenvironment may contribute to the high risk of tumor recurrence following conventional therapies, such as sorafenib, PD-1 antagonists and PD-L1 antagonists.

Provided herein is a method of treating HCC in a subject, comprising administering a therapeutically effective amount of an anti-Claudin-1 antibody. In some aspects, the HCC is resistant to sorafenib therapy. In some aspects, the HCC is resistant to a PD-1 antagonist therapy (e.g. nivolumab). In some aspects, the HCC is resistant to PD-L1 antagonist therapy. This is based in part on the observation that administration of exemplary anti-Claudin-1 antibodies were efficacious at decreasing cell viability of HCC cells derived from sorafenib resistant HCC and nivolumab resistant HCC. Without being bound by theory, such resistant HCC cells may exhibit high expression of Claudin-1, making them targetable and susceptible to anti-Claudin-1 antibody therapies. In some instances, the high expression of Claudin-1 may be pharmacologically induced. Accordingly, this provides an advantageous property that corresponds with a high therapeutic potential for the use of anti-Claudin-1 antibodies to treat subsets of HCC that are resistant to conventional therapies, such as sorafenib and nivolumab.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Exemplary methods and materials are described herein, although methods and materials similar or equivalent to those described herein can be used in practice or testing of the present disclosure. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In some aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “treat,” “treating” and “treatment” are interchangeable, and encompasses partially or completely preventing, ameliorating, mitigating and/or managing a symptom, a secondary disorder or a condition associated with hepatocellular carcinoma (HCC). In some aspects, the HCC is metastatic HCC, unresectable HCC, refractory HCC, or relapsed HCC. In some embodiments, the HCC is any of early stage HCC, non-metastatic HCC, primary HCC, advanced HCC, locally advanced HCC, metastatic HCC, HCC in remission, recurrent HCC, HCC in an adjuvant setting, or HCC in a neoadjuvant setting. In some aspects, the hepatocellular carcinoma is relapsed, refractory or resistant to conventional therapy. The term “treating” as used herein refers to application or administration of one or more active agents to a subject, who has a symptom, a secondary disorder or a condition associated with HCC, with the purpose to partially or completely alleviate, ameliorate, relieve, delay onset of, inhibit progression of, reduce severity of, and/or reduce incidence of one or more symptoms, secondary disorders or features associated with HCC. Symptoms, secondary disorders, and/or conditions associated with HCC include, but are not limited to, abdominal pain, fatigue, anorexia, cachexia, ascites, and biliary obstruction. Treatment may be administered to a subject who exhibits only early signs of such symptoms, disorder, and/or condition for the purpose of decreasing the risk of developing the symptoms, secondary disorders, and/or conditions associated with HCC.

The term human “Claudin-1” (or “CLDN1”) refers to a protein having the sequence shown in NCBI Accession Number NP_066924.1, or any naturally occurring variants commonly found in HCV permissive human populations.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×10⁷ M¹, and binds to the predetermined antigen with an affinity that is at least two-fold greater than the affinity for binding to a non-specific antigen (e.g., BSA, casein).

The term “antigen binding fragment”, “antigen binding region” or “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” Various methods are known in the art for numbering the amino acids sequences of antibodies and identification of the complemenatry determining regions. For example, the Kabat numbering system (See Kabat, E. A., et al., Sequences of Protein of immunological interest, Fifth Edition, US Department of Health and Human Services, US Government Printing Office (1991)) or the IMGT numbering system (See IMGT®, the international ImMunoGeneTics information system®. Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M. P. et al., Biomolecules, 2014 December; 4(4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M. P. et al., Biomolecules, 2014 December; 4(4), 1102-1139).

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains, in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

The term “humanized” refers to sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

It is understood that wherever aspects are described herein with the language “comprising,” otherwise analogous aspects described in terms of “consisting of” and/or “consisting essentially of” are also provided.

As used herein, the term “administering” refers to the physical introduction of a composition comprising a therapeutic agent (e.g., an anti-Claudin-1 antibody) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-Claudin-1 antibody clinically proven to affect a significant decrease in cancer or slowing of progression of cancer, such as HCC.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “tumor” as used herein refers to any mass of tissue that results from excessive cell growth or proliferation, either benign (non-cancerous) or malignant (cancerous), including pre-cancerous lesions.

“Sorafenib” or 4-[4-[[4-chloro-3-(trifluoromethyl)phenyl]carbamoylamino]phenoxy]-N -methyl-pyridine-2-carboxamide (e.g., PubChem ID 216239), is an approved drug for the treatment of hepatocellular carcinoma (HCC). The mechanism of action is mediated by the inhibitory activity exerted on kinases overexpressed in a series of molecular pathways involved in the transformation of normal cells into tumor cells, in particular receptors with kinase activity and on Raf kinases.

“Nivolumab” (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

“Antagonist” and “inhibitor” are used interchangeably herein and include any substance that interferes with or inhibits the physiological action of another.

The term “high expression of Claudin-1” refers to the proportion of cells in a test tissue sample that is scored as expressing Claudin-1. In some aspects, Claudin-1 expression is assayed by immunohistochemistry (IHC), where the high expression of Claudin-1 of a sample means that at least about 50%, at least about 60%, at least about 70%, at least about 80%, at least about 90%, or 100% of the total number of cells in the test sample express Claudin-1.

A “patient” as used herein includes any patient who is afflicted with a cancer (e.g., a fibrotic cancer). The terms “subject” and “patient” are used interchangeably herein.

### II. Anti-Claudin-1 Antibodies

The present disclosure provides the use of an anti-Claudin-1 antibody, or antigen binding fragment thereof, for treating drug-resistant HCC. In some aspects, the HCC is sorafenib resistant. In some aspects, the HCC is resistant to a PD-1 antagonist. In some aspects, the PD-1 antagonist is nivolumab.

Antibodies directed against human Claudin-1 have been previously described to treat hepatitis C virus infection, hepatocellular carcinoma, and certain fibrotic diseases, such as lung fibrosis (see WO 2010/034812, WO 2016/146809, and WO 2021/094469).

Anti-Claudin-1 antibodies that can be used in the practice of the present disclosure include any antibody raised against Claudin-1. Exemplary anti-Claudin-1 antibodies include, but are not limited to those described in in WO 2010/034812 and WO 2017/162678, the contents of each of which are incorporated herein by reference. Exemplary anti-Claudin-1 antibodies include, but are not limited to those described in European Patent No. EP 1,167,389, U.S. Pat. No. 6,627,439, PCT Application No. WO 2014/132307, WO 2015/014659, No. WO 2015/014357, the contents of each of which are incorporated herein by reference. Exemplary anti-Claudin-1 antibodies are also described in Yamashita et al., J. Pharmacol. Exp. Ther., 2015, 353(1): 112-118.

In some aspects, anti-Claudin-1 antibodies are polyclonal antibodies or monoclonal antibodies. In some aspects, anti-Claudin-1 antibodies are humanized antibodies.

Exemplary anti-Claudin-1 antibody or antigen binding fragments of the disclosure are described in Table 1. In some aspects, the anti-Claudin-1 antibody is “H1L1”. In some aspects, the anti-Claudin-1 antibody is “H3L3”. The CDRs shown in Table 1 is defined according to the IMGT nomenclature (See IMGT®, the international ImMunoGeneTics information system®. Available online: http://www.imgt.org/).

TABLE 1
Anti-Claudin-1 antibody sequences of the disclosure

| | | SEQ ID |
|---|---|---|
| Description | Sequence | NO: |
| H1L1- | EVQLVESGGGLVKPGGSLRLSCAASGFSFSSYGMNWVRQA | 1 |
| Heavy Chain | PGKGLEWVSSISPSGSYFYYADSVKGRFTISRDNAKNSLYLQ | |
| | MNSLRAEDTAVYYCARLPGFNPPFDHWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPELLGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |
| H1L1- | DIQMTQSPATLSVSPGERATLSCKASQNVGGNVDWYQWKP | 2 |
| Light Chain | GQAPRLLIYGASNRYTGIPARFRGSGSGTEFTLTISSLQSEDF | |
| | AVYYCLQYKNNPWTFGQGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| H1L1-VH | EVQLVESGGGLVKPGGSLRLSCAASGFSFSSYGMNWVRQA | 3 |
| | PGKGLEWVSSISPSGSYFYYADSVKGRFTISRDNAKNSLYLQ | |
| | MNSLRAEDTAVYYCARLPGFNPPFDHWGQGTLVTVSS | |
| H1L1-VL | DIQMTQSPATLSVSPGERATLSCKASQNVGGNVDWYQWKP | 4 |
| | GQAPRLLIYGASNRYTGIPARFRGSGSGTEFTLTISSLQSEDF | |
| | AVYYCLQYKNNPWTFGQGTKVEIK | |
| H1L1/H3L3- | GFSFSSYG | 5 |
| CDR H1 | | |
| H1L1/H3L3- | ISPSGSYF | 6 |
| CDR H2 | | |
| H1L1/H3L3- | ARLPGFNPPFDH | 7 |
| CDR H3 | | |
| H1L1/H3L3- | QNVGGN | 8 |
| CDR L1 | | |
| H1L1/H3L3- | GA | — |
| CDR L2 | | |
| H1L1/H3L3- | LQYKNNPWT | 10 |
| CDR L3 | | |
| H3L3- | QVQLVESGGGVVQPGRSLRLSCLGSGFSFSSYGMNWVRQA | 11 |
| Heavy Chain | PGKGLEWVASISPSGSYFYYADSVKGRFTISRDNSKNTLYLQ | |
| | MTSLRAEDTAIYYCARLPGFNPPFDHWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKRVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSN | |
| | KALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPG | |
| H3L3- | DIQMTQSPSSLSASVGDRVTITCKASQNVGGNVDWYQWKP | 12 |
| Light Chain | GKAPKLLIYGASNRYTGVPDRFRGSGSGTDFTLTISSLQPED | |
| | VATYYCLQYKNNPWTFGGGTKVEIKRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| H3L3-VH | QVQLVESGGGVVQPGRSLRLSCLGSGFSFSSYGMNWVRQA | 13 |
| | PGKGLEWVASISPSGSYFYYADSVKGRFTISRDNSKNTLYLQ | |
| | MTSLRAEDTAIYYCARLPGFNPPFDHWGQGTLVTVSS | |
| H3L3-VL | DIQMTQSPSSLSASVGDRVTITCKASQNVGGNVDWYQWKP | 14 |
| | GKAPKLLIYGASNRYTGVPDRFRGSGSGTDFTLTISSLQPED | |
| | VATYYCLQYKNNPWTFGGGTKVEIK | |

In some aspects, the “H1L1” anti-Claudin-1 antibody comprises a complementarity determining region (CDR) CDRH1 comprising the amino acid sequence set forth in SEQ ID NO: 5, a CDR H2 comprising the amino acid sequence set forth in SEQ ID NO: 6, a CDR H3 comprising the amino acid sequence set forth in SEQ ID NO: 7, a CDR L1 comprising the amino acid sequence set forth in SEQ ID NO: 8, a CDR L2 comprising the amino acid sequence GA, and a CDR L3 comprising the amino acid sequence set forth in SEQ ID NO: 10.

In some aspects, the “H1L1” anti-Claudin-1 antibody or antigen binding fragment thereof comprises a heavy chain variable region comprising an amino acid sequence of SEQ ID NO: 3 and a light chain variable region comprising an amino acid sequence of SEQ ID NO: 4.

In some aspects, the “H1L1” anti-Claudin-1 antibody or antigen binding fragment thereof comprises a heavy chain comprising an amino acid sequence of SEQ ID NO: 1 and a light chain comprising an amino acid sequence of SEQ ID NO: 2.

In some aspects, the “H3L3” anti-Claudin-1 antibody comprises a complementarity determining region (CDR) CDRH1 comprising the amino acid sequence set forth in SEQ ID NO: 5, a CDR H2 comprising the amino acid sequence set forth in SEQ ID NO: 6, a CDR H3 comprising the amino acid sequence set forth in SEQ ID NO: 7, a CDR L1 comprising the amino acid sequence set forth in SEQ ID NO: 8, a CDR L2 comprising the amino acid sequence GA, and a CDR L3 comprising the amino acid sequence set forth in SEQ ID NO: 10.

In some aspects, the “H3L3” anti-Claudin-1 antibody or antigen binding fragment thereof comprises a heavy chain variable region comprising an amino acid sequence of SEQ ID NO: 13 and a light chain variable region comprising an amino acid sequence of SEQ ID NO: 14.

In some aspects, the “H3L3” anti-Claudin-1 antibody or antigen binding fragment thereof comprises a heavy chain comprising an amino acid sequence of SEQ ID NO: 11 and a light chain comprising an amino acid sequence of SEQ ID NO: 12.

In some aspects, the six complementarity determining regions (CDRs) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain variable region (“VH”) and the light chain variable region (“VL”) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain and light chain of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody may be a full monoclonal antibody having an isotope selected from the group consisting of IgG1, IgG2, IgG3 and IgG4. In some aspects, the anti-Claudin-1 antibody may be a fragment of a monoclonal antibody selected from the group consisting of Fv, Fab, F(ab′)₂, Fab′, dsFv, scFv, sc(Fv)₂ and diabodies.

In some aspects, the anti-Claudin-1 antibodies (or biologically active variants or fragments thereof) suitable for use according to the present disclosure may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, NY; and Wilchek and Bayer, Anal. Biochem., 1988, 171:1-32). Preferably, molecular entities are attached at positions on the antibody molecule that do not interfere with the binding properties of the resulting conjugate, e.g., positions that do not participate in the specific binding of the antibody to its target.

In some aspects, the antibody molecule (e.g. anti-Claudin-1 antibody) and molecular entity may be covalently, directly linked to each other. In some aspects, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present disclosure is conjugated to a detectable agent. Any of a wide variety of detectable agents can be used, including, without limitation, various ligands, radionuclides (e.g., ³H, ¹²⁵I, ¹³¹I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and aequorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present disclosure (or a biologically active fragment thereof) include, but are not limited to, linear or branched hydrophilic polymeric groups, fatty acid groups, or fatty ester groups.

In some aspects, the anti-Claudin-1 antibodies are full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)2 fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent or a detectable agent. The anti-Claudin-1 antibody-related molecules according to the present disclosure retain the ability to bind its antigen (e.g. Claudin-1), in particular the extracellular domain of Claudin-1.

### III. Methods of Use

This disclosure provides methods of administrating an effective amount of an anti-Claudin-1 antibody, or an antigen binding fragment thereof, or of a pharmaceutical composition thereof, to a subject in need thereof (i.e., a subject having hepatocellular carcinoma). This disclosure provides a method of inhibiting a hepatocellular carcinoma (HCC) in a subject, comprising administering an anti-Claudin-1 antibody, or antigen binding fragment thereof to the subject.

Methods of the present disclosure may be accomplished using an effective amount of an anti-Claudin-1 antibody, or an antigen binding fragment thereof, or a pharmaceutical composition comprising such an antibody or fragment.

In some aspects, the methods provided herein are used to treat a subject having sorafenib and/or nivolumab resistant HCC. Sorafenib remains the globally accepted systemic first-line treatment for advanced HCC (Llovet J. M., et al. N. Engl. J. Med. 2008; 359:378-390). Even though it only has modest improvement in over-all median survival, its approval in 2007 is one of the hallmarks of HCC treatment. Sorafenib is a molecularly-targeted agent that works on the vascular endothelial growth factor receptors (VEGFR1, 2, 3), platelet-derived growth factor receptor-β(PDGFRβ) and the Raf family kinases (predominantly C-Raf rather than B-Raf) (Liu L., et al., Cancer Res. 2006; 66:11851-11858). However, it has been observed that in a large number of patients, its effectiveness is hampered by drug resistance. Since HCC is highly heterogeneous, within the tumor and among individuals, this influences disease progression, classification, prognosis, and naturally cellular susceptibility to drug resistance.

Nivolumab is a fully human IgG4 anti-programmed death-1 (PD-1) monoclonal antibody that inhibits immune checkpoint signaling. Nivolumab treatment improves survival compared with chemotherapy across several tumor types, including melanoma, non-small cell lung cancer, and renal cell carcinoma. Nivolumab is also approved in various countries for patients with advanced HCC who were previously treated with sorafenib.

This disclosure provides a method of treating HCC in a subject, comprising administering an anti-Claudin-1 antibody, or antigen binding fragment thereof to a subject. This disclosure also provides a method of treating sorafenib-resistant HCC in a subject, comprising administering an anti-Claudin-1 antibody, or antigen binding fragment thereof to a subject. This disclosure also provides a method of treating anti-PD-1 resistant HCC in a subject, comprising administering an anti-Claudin-1 antibody, or antigen binding fragment thereof. This disclosure also provides a method of treating anti-PD-L1 resistant HCC in a subject, comprising administering an anti-Claudin-1 antibody, or antigen binding fragment thereof.

In some aspects, the anti-Claudin-1 antibody or antigen binding fragment thereof is administered simultaneously or sequentially with sorafenib to the subject. In some aspects, the anti-Claudin-1 antibody, or antigen binding fragment thereof, is administered after administration of sorafenib, a PD-1 antagonist or a PD-L1 antagonist.

In some aspects, the subject was previously treated with a PD-1 antagonist.

In some aspects, the PD-1 antagonist is a small molecule PD-1 inhibitor. In some aspects, the PD-1 antagonist is an anti-PD-1 antibody or antigen binding fragment thereof. In some aspects, the PD-1 antagonist is selected from the group consisting of nivolumab, pembrolizumab, cemiplimab, and dostarlimab.

In some aspects, the subject was previously treated with a PD-L1 antagonist.

In some aspects, the PD-L1 antagonist is a small molecule PD-L1 inhibitor. In some aspects, the PD-L1 antagonist is a anti-PD-L1 antibody or antigen binding fragment thereof. In some aspects, the PD-L1 antagonist is selected from the group consisting of atezolizumab (TECENTRIQ; RG7446; MPDL3280A; RO5541267), durvalumab (MEDI4736), BMS-936559, avelumab (bavencio), LY3300054, CX-072 (Proclaim-CX-072), FAZ053, KN035, and MDX-1105.

In some aspects, the HCC is characterized by a high expression of Claudin-1. In some aspects, the methods disclosed herein further comprise detecting Claudin-1 expression levels in a HCC tumor sample from a subject. In some aspects, the methods disclosed herein further comprise comparing the Claudin-1 expression levels to the expression level of Claudin-1 in a reference sample, wherein if the expression levels of Claudin-1 in the HCC tumor sample are increased with respect to the expression levels of Claudin-1 in a reference sample, then the subject is administered the anti-Claudin-1 antibody, or the anti-Claudin-1 antibody and sorafenib.

In some aspects, the Claudin-1 expression levels are quantified via immunohistochemistry (IHC) test. In some aspects, the IHC test is graded on a score of 0 to +3. In some aspects, a high expression of Claudin-1 as graded by the IHC test is +1, +2, or +3.

In order to improve the treatment of HCC, in some aspects, the present disclosure provides identifying a patient as having a high expression of Claudin-1 and providing an anti-Claudin-1 antibody, or antigen binding fragment thereof, or an anti-Claudin-1 antibody, or antigen binding fragment thereof and sorafenib.

In some aspects, the present disclosure is directed to identifying a patient as having a HCC with high expression of Claudin-1 and treating said HCC by administering a combination of an anti-Claudin-1 antibody, or antigen binding fragment thereof, or an anti-Claudin-1 antibody, or antigen binding fragment thereof and sorafenib.

In some aspects, the disclosure includes a method of selecting a HCC patient for immunotherapy, comprising: (a) determining the level of Claudin-1 expression in a tumor sample; and (b) selecting the tumor for immunotherapy if the tumor sample has high expression of Claudin-1. In some aspects, the immunotherapy is administration of an anti-Claudin-1 antibody, or antigen binding fragment thereof, or an anti-Claudin-1 antibody, or antigen binding fragment thereof and sorafenib.

In some aspects, the disclosure includes a method of identifying a HCC tumor in a human patient as eligible for immunotherapy, comprising: (a) determining the level of Claudin-1 expression in a tumor sample; and (b) identifying the tumor as eligible for immunotherapy if the tumor sample has high expression of Claudin-1. In some aspects, the immunotherapy is administration of an anti-Claudin-1 antibody, or antigen binding fragment thereof, or an anti-Claudin-1 antibody, or antigen binding fragment thereof and sorafenib.

In some aspects, the disclosure includes a method of identifying a HCC tumor in a human patient as eligible for immunotherapy, comprising: (a) determining if a tumor is resistant to sorafenib therapy; and (b) identifying the tumor as eligible for immunotherapy if the tumor sample is resistant to sorafenib treatment. In some aspects, the immunotherapy is administration of an anti-Claudin-1 antibody, or antigen binding fragment thereof, or an anti-Claudin-1 antibody, or antigen binding fragment thereof and sorafenib.

In some aspects, the disclosure includes a method of identifying a HCC tumor in a human patient as eligible for immunotherapy, comprising: (a) determining if a tumor is resistant to PD-1 antagonist therapy; and (b) identifying the tumor as eligible for immunotherapy if the tumor sample is resistant to nivolumab treatment. In some aspects, the immunotherapy is administration of an anti-Claudin-1 antibody, or antigen binding fragment thereof, or an anti-Claudin-1 antibody, or antigen binding fragment thereof and sorafenib.

Cancers may be diagnosed as sorafenib and/or PD-1 antagonist resistant after treatment with sorafenib and/or PD-1 antagonist has commenced. Alternatively, cancers may be diagnosed as sorafenib and/or PD-1 antagonist resistant prior to initiation of treatment with such compounds. Sorafenib and/or PD-1 antagonist resistance in the tumor may occur after, e.g., 6 months or longer of sorafenib and/or PD-1 antagonist treatment. Alternatively, sorafenib and/or PD-1 antagonist resistance of the tumor may be diagnosed less than 6 months after sorafenib and/or PD-1 antagonist treatment has commenced.

Diagnosis of sorafenib and/or PD-1 antagonist resistance may be accomplished by way of monitoring tumor progression during sorafenib and/or PD-1 antagonist treatment. Tumor progression may be determined by comparison of tumor status between time points after treatment has commenced or by comparison of tumor status between a time point after treatment has commenced to a time point prior to initiation of sorafenib and/or PD-1 antagonist treatment. Tumor progression may be monitored during sorafenib and/or PD-1 antagonist treatment visually, for example, by means of radiography, for example, X-ray, CT scan, or other monitoring methods known to the skilled artisan, including palpitation of the cancer or methods to monitor tumor biomarker levels. Progression of the cancer during treatment with sorafenib and/or PD-1 antagonist indicates sorafenib and/or PD-1 antagonist resistance. Detection of new tumors or detection of metastasis indicates tumor progression. Cessation of tumor shrinkage indicates tumor progression. Growth of the cancer is indicated by, for example, increase in tumor size, metastasis or detection of new cancer, and/or a rise in tumor biomarker levels.

In some embodiments, the healthy/control/reference sample is a sample from a normal tissue. In some embodiments, the normal tissue is a tissue that is an adjacent tissue to the cancer in the subject. For example, when assaying for expression via staining, the levels of the protein are above those understood to be “negative” to one of skill in the art for the particular assay. Exemplary assays include but are not limited to RNA based assays, FACS, and IHC, each with their appropriate levels of positive and negative results. Any method of detecting the level of a protein in a sample is contemplated. One skilled in the art can select a suitable method depending on the type of sample being analyzed and the identity and number of proteins being detected. Nonlimiting exemplary such methods include immunohistochemistry, ELISA, Western blotting, multiplex analyte detection (using, for example, Luminex technology), mass spectrometry, etc. Similarly, any method of detecting the level of an mRNA in a sample is contemplated. One skilled in the art can select a suitable method depending on the type of sample being analyzed and the identity and number of mRNAs being detected. Nonlimiting exemplary such methods include RT-PCR, quantitative RT-PCR and microarray-based methods, etc.

In some aspects, Claudin-1 expression is determined by receiving the results of an assay capable of determining Claudin-1 expression.

In order to assess the Claudin-1 expression, in some aspects, a test tissue sample is obtained from the patient who is in need of the therapy. In some aspects, a test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen or cryoconserved tissue sample. In some aspects, the test tissue sample is a fresh frozen (FF) tissue (e.g., tumor) sample. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In some aspects, the assessment of Claudin-1 expression can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express Claudin-1 on the surface of the cells based on an assessment that the proportion of cells in the test tissue sample that express Claudin-1 on the cell surface is higher than a predetermined threshold level.

In any of the methods comprising the measurement of Claudin-1 expression in a test tissue sample, however, it should be understood that the step comprising the provision of a test tissue sample obtained from a patient is an optional step. In some aspects the method includes this step, and in other aspects, this step is not included in the method. It should also be understood that in some aspects the “measuring” or “assessing” step to identify, or determine the number or proportion of, cells in the test tissue sample that express Claudin-1 is performed by a transformative method of assaying for Claudin-1 expression, for example by performing a reverse transcriptase-polymerase chain reaction (RT-PCR) assay or an IHC assay. In some aspects, no transformative step is involved and Claudin-1 expression is assessed by, for example, reviewing a report of test results from a laboratory. In some aspects, Claudin-1 expression is assessed by reviewing the results of an immunohistochemistry assay from a laboratory. In some aspects, the steps of the methods up to, and including, assessing Claudin-1 expression provides an intermediate result that may be provided to a physician or other healthcare provider for use in selecting a suitable candidate for the combination therapy of a Claudin-1 inhibitor and an immune checkpoint inhibitor. In some aspects, the steps of the methods up to, and including, assessing Claudin-1 expression provides an intermediate result that may be provided to a physician or other healthcare provider for use in selecting a suitable candidate for an immune checkpoint inhibitor therapy. In some aspects, the steps that provide the intermediate result is performed by a medical practitioner or someone acting under the direction of a medical practitioner. In some aspects, these steps are performed by an independent laboratory or by an independent person such as a laboratory technician.

In some aspects of any of the present methods, the proportion of cells that express Claudin-1 is assessed by performing an assay to detect the presence of Claudin-1 RNA. In some aspects, the presence of Claudin-1 RNA is detected by RT-PCR, in situ hybridization or RNase protection. In some aspects, the presence of Claudin-1 RNA is detected by an RT-PCR based assay. In some aspects, scoring the RT-PCR based assay comprises assessing the level of Claudin-1 RNA expression in the test tissue sample relative to a predetermined level.

In some aspects, the proportion of cells that express Claudin-1 is assessed by performing an assay to detect the presence of Claudin-1 polypeptide. In some aspects, the presence of Claudin-1 polypeptide is detected by IHC, enzyme-linked immunosorbent assay (ELISA), in vivo imaging, or flow cytometry. In some aspects, Claudin-1 expression is assayed by IHC. In some aspects of all of these methods, cell surface expression of Claudin-1 is assayed using, e.g., IHC or in vivo imaging.

In some aspects, the immunohistochemistry assay is scored at a low magnification. In some aspects, low magnification is about 20×. In some aspects, the immunohistochemistry assay is scored at high magnification. In some aspects, high magnification is about 40×.

In some aspects, the immunohistochemistry assay is scored by an image analysis software. In some aspects, the immunohistochemistry assay is scored by pathologist visual immune score. In some aspects, the immunohistochemistry assay is scored manually.

### IV. PD-1 and PD-L1 Antagonists

Immune checkpoint proteins, for example PD-1 and PD-L1, interact with specific ligands which send a signal into T-cells that inhibits T-cell function. Cancer cells exploit this by driving high level expression of checkpoint proteins on their surface thereby suppressing the anti-cancer immune response.

In some aspects, the PD-1 antagonist is an antibody. In some aspects, the PD-1 antagonist is an antibody or fragment thereof that specifically binds to PD-1.

In some aspects, the subject having HCC is resistant to treatment with a PD-1 antagonist. In some aspects, the HCC is resistant to anti-PD-1 antibody therapy. In some aspects, the anti-PD-1 antibody is nivolumab. In some aspects, the subject having HCC resistant to treatment with a PD-1 antagonist is administered an anti-Claudin-1 antibody or antigen binding fragment thereof.

In some aspects, the HCC is treated with a pharmaceutical composition comprising an anti-Claudin-1 antibody, or antigen binding fragment thereof.

The complete nucleotide and amino acid sequences of human PD-1 can be found under GenBank Accession No. NG_012110.1 and NP_005009.2. In some aspects, the anti-PD-1 antibody is nivolumab. Nivolumab (also known as “OPDIVO®”; BMS-936558; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In some aspects, the anti-PD-1 antibody or fragment thereof cross-competes with nivolumab. In some aspects, the anti-PD-1 antibody or fragment thereof binds to the same epitope as nivolumab. In certain aspects, the anti-PD-1 antibody has the same CDRs as nivolumab.

In some aspects, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587, the contents of which are incorporated by reference herein in their entireties.

Other exemplary anti-PD-1 antibodies include, but are not limited to, monoclonal antibodies 5C4 (referred to herein as Nivolumab or BMS-936558), 17D8, 2D3, 4H1, 4A11, 7D3, and 5F4, described in WO 2006/121168, the contents of which are incorporated by reference herein in its entirety. Other exemplary anti-PD-1 antibodies include, but are not limited to lambrolizumab (MK-3475) described in WO 2008/156712, and AMP-514 described in WO 2012/145493, the contents of which are incorporated herein by reference in their entireties. Exemplary anti-PD-1 antibodies and other PD-1 inhibitors include, but are not limited to those described in WO 2009/014708, WO 03/099196, WO 2009/114335 and WO 2011/161699, the contents of which are incorporated herein by reference in their entireties. In some aspects, the anti-PD-1 antibody is REGN2810. In some aspects, the anti-PD-1 antibody is PDR001. In some aspects, the anti-PD-1 antibody is pidilizumab (CT-011).

Other exemplary anti-PD-1 monoclonal antibodies include but are not limited to those that have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810;see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540), the contents of each of which are incorporated herein by reference in their entireties.

In some aspects, the anti-PD-1 antibody or antigen binding fragment thereof cross-competes with pembrolizumab. In some aspects, the anti-PD-1 antibody or antigen binding fragment thereof binds to the same epitope as pembrolizumab. In some aspects, the anti-PD-1 antibody or antigen binding fragment thereof has the same CDRs as pembrolizumab. In some aspects, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In some aspects, the PD-1 antagonist is selected from the group consisting of nivolumab, pembrolizumab, cemiplimab, and dostarlimab.

In some aspects, the PD-L1 antagonist is an antibody. In some aspects, the PD-L1 antagonist is an antibody or fragment thereof that specifically binds to PD-L1.

In some aspects, the subject having HCC is resistant to treatment with a PD-L1 antagonist. In some aspects, the HCC is resistant to anti-PD-L1 antibody therapy. In some aspects, the anti-PD-L1 antibody is nivolumab. In some aspects, the subject having HCC resistant to treatment with a PD-L1 antagonist is administered an anti-Claudin-1 antibody or antigen binding fragment thereof.

Exemplary PD-L1 antagonists include, but are not limited to, BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)).

As the skilled person will know, alternative and/or equivalent names may be in use for certain antibodies mentioned above. Such alternative and/or equivalent names are interchangeable in the context of the present disclosure.

### V. Administration

The anti-Claudin-1 antibody can be administered to a subject in need thereof by any suitable route. In some aspects, the anti-Claudin-1 antibody is administered in combination with sorafenib, as described above. Various delivery systems are known and can be used to administer the antibodies or sorafenib, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, and oral routes. An anti-Claudin-1 antibody may be administered by any convenient or other appropriate route, for example, by infusion or bolus injection, by absorption through epithelial or mucosa linings (e.g., oral mucosa, bronchial mucosa, rectal and intestinal mucosa, etc.). Administration can be systemic or local.

As will be appreciated by those of ordinary skill in the art, in aspects where an antibody is administered in combination with an additional therapeutic agent (e.g., sorafenib), the antibody and therapeutic agent may be administered by the same route (e.g., intravenously) or by different routes (e.g., intravenously, orally, or subcutaneously). Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the bispecific antibody and administration of an additional therapeutic agent occur within about one month, or within about one, two or three weeks, or within about one, two, three, four, five, or six days, of each other.

### VI. Kits

The present disclosure provides a pharmaceutical pack or kit comprising one or more containers (e.g., vials, ampoules, test tubes, flasks or bottles) containing one or more ingredients of an inventive pharmaceutical composition, allowing administration of an anti-Claudin-1 antibody and/or sorafenib.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kits will preferably be maintained in close confinement for commercial sale.

In some aspects, a pharmaceutical pack or kit includes one or more additional therapeutic agent(s) as described above. Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

### EXAMPLES

Any examples provided herein are offered by way of illustration and not by way of limitation. The following examples are illustrative and do not limit the scope of the claimed aspects.

### Example 1. Perturbation Studies in HCC Patient-Derived Spheroids

Fragments of tumor tissue (5×5 mm to 8×8 mm size) were excised from tumor mass of HCC patients (n=15 different donors, Table 2) and then processed with enzymatic and mechanical dissociation to generate clusters of tumor cells similarly as described recently (Crouchet E, et al., Nat Commun 2021; 12:5525; Jühling F, et al., Gut 2021; 70:157-69). Aggregates of cells were assembled into spheroids in 96-well Black/Clear Bottom Low Flange Ultra-Low Attachment Microplates (Corning) and cultured in MammoCult basal medium (STEMCELL Technologies), supplemented with human proliferation supplement (3.4%), hydrocortisone (0.056%), heparin (0.011%) and 50% patients' own serum.

Tumorspheres were treated for 6 days with 10 μg/mL isotype control mAb, with 10 μg/mL anti-Claudin-1 mAb “H3L3” (Table 1), with sorafenib (10 μM, Selleckchem) or with Nivolumab (10 μg/mL, Selleckchem) with at least duplicates per condition. Tumorsphere viability was assessed at day 6 using CellTiterGlo 3D (Promega) according to the manufacturer's instructions.

TABLE 2
Clinical characteristics of HCC patients, recruited
for HCC spheroid perturbation studies

| | Etiology of | | |
|---|---|---|---|
| | chronic liver | Fibrosis | |
| # | disease | stage* | Histological characteristics^{#} |
| ST1 | hemochromatosis | F4 | G1, no invasion, pT1b |
| S06 | HCV1b, SVR | A2F2 | — |
| S07 | ASH | — | — |
| S15 | HCV1a, SVR | A0F2 | G2, vascular invasion pT2 |
| S17 | HCV1a, SVR | A0F4 | G2, no vascular invasion pT2 |
| S18 | HCV | F2A2 | G2-3, vascular invasion |
| S24 | — | — | — |
| 381 | NASH | F1-2, A1-A2 | G2, vascular invasion, pT2 |
| S19 | NASH | F3-4 | G2, vascular invasion |
| S22 | NASH | F3 | G1-2, vascular invasion, pT2 |
| S349 | HCV | F1 | G1, vascular invasion, pT1b |
| S394 | No chronic liver | F0-1/A0 | G4, poorly differentiated, |
| | disease | | vascular invasion |
| S13 | NASH | — | — |
| S16 | ASH | F4 | G2-G3, vascular invasion, pT2 |
| R2 | ASH | F3 | — |
| R21 | ASH | F4 | — |
| R11 | HBV | F4 | — |
| 462 | NASH | F4 | G1, no vascular invasion |

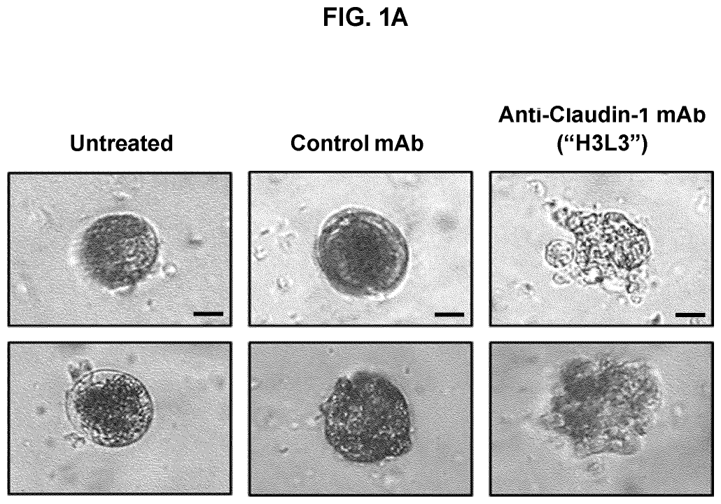

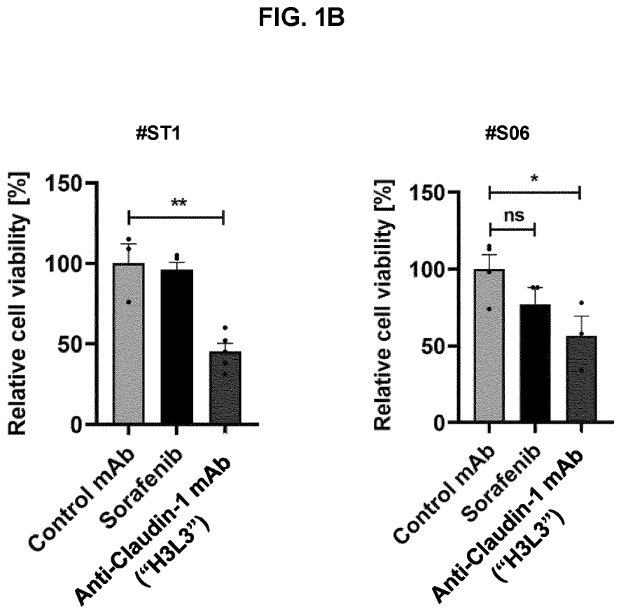

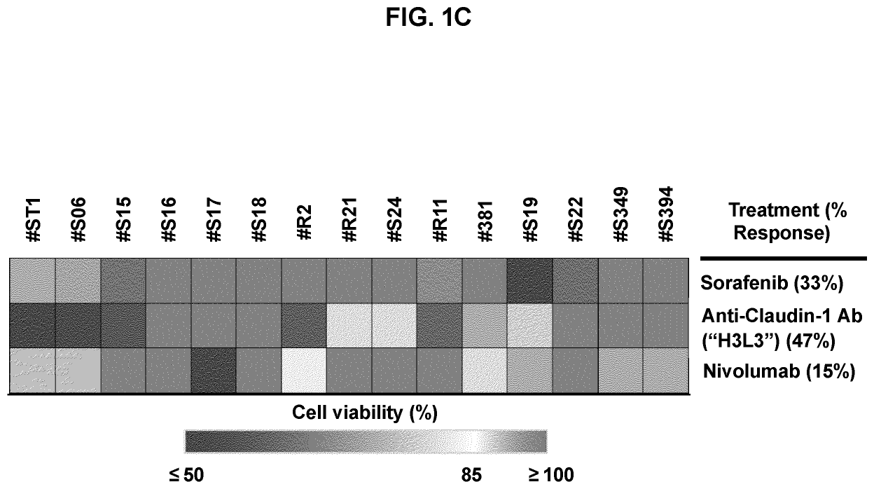

The effect of an anti-Claudin-1 antibody (anti-Claudin-1 mAb “H3L3”) on tumor growth in a fully patient-derived culture system, modeling tumor heterogeneity was also assessed. Cultured as multicellular micro-tissues, primary HCC tumorspheres maintain original cell-cell contacts and recapitulate non-parenchymal cells of the TME, including T-cells, which are relevant for tumor progression and therapeutic resistance (Song Y, et al., J Exp Clin Cancer Res 2018; 37:109). Anti-Claudin-1 mAb “H3L3” treatment markedly disrupted the architecture of HCC spheroids (FIG. 1A). Moreover, anti-Claudin-1 mAb “H3L3” showed a pronounced effect on sorafenib-resistant HCC spheroid cell viability (p=0.003 and p=0.04, Student's t-test, FIG. 1B). A subsequent screen in HCC spheroids derived from 15 different HCC patients (patients' characteristics shown in Table 2), corroborated the effects of anti-Claudin-1 mAb “H3L3” on tumor cell viability with superior response rates compared to sorafenib and nivolumab (47% vs. 33% and 15%, defined as a mean decrease in cell viability of >15%, FIG. 1C).

Collectively, these data indicate strong suppressive effects of anti-Claudin-1 mAb “H3L3” on HCC growth including sorafenib-and nivolumab-resistant tumors.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

### INCORPORATION BY REFERENCE

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### EMBODIMENTS

Additional embodiments of the disclosure include the following:

Embodiment 1. A method of treating hepatocellular carcinoma (HCC) in a subject, comprising administering a therapeutically effective amount of an anti-Claudin-1 antibody, wherein the HCC is resistant to sorafenib therapy.

Embodiment 2. A method of treating sorafenib-resistant hepatocellular carcinoma in a subject, comprising administering a therapeutically effective amount of an anti-Claudin-1 antibody, wherein the HCC is resistant to a PD-1 antagonist therapy.

Embodiment 3. The method of embodiment 1, wherein the subject was previously treated with sorafenib.

Embodiment 4. The method of embodiment 2, wherein the subject was previously treated with a PD-1 antagonist.

Embodiment 5. The method of embodiments 2 or 4, wherein the PD-1 antagonist is an antibody.

Embodiment 6. The method of embodiment 5, wherein the PD-1 antagonist selected from the group consisting of nivolumab, pembrolizumab, cemiplimab, and dostarlimab.

Embodiment 7. The method of embodiment 6, wherein the PD-1 antagonist is nivolumab.

Embodiment 8. The method of any one of embodiments 1-7, wherein the HCC is characterized by a high expression of Claudin-1 relative to a reference sample, or resistance to sorafenib and/or nivolumab.

Embodiment 9. The method of embodiment 8, wherein the reference sample is a tissue sample from a normal tissue, wherein the normal tissue is adjacent to a HCC tumor.

Embodiment 10. The method of any one embodiments 1-9, wherein the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

Embodiment 11. The method of any one of embodiments 1-10, wherein the anti-Claudin-1 antibody is humanized.

Embodiment 12. The method of any one of embodiments 1-11, wherein the anti-Claudin-1 antibody comprises a VH comprising the amino acid sequence set forth in SEQ ID NO: 3 or SEQ ID NO: 13.

Embodiment 13. The method of any one of embodiments 1-12, wherein the anti-Claudin-1 antibody comprises a VL comprising the amino acid sequence set forth in SEQ ID NO: 4 or SEQ ID NO: 14.

Embodiment 14. The method of any one of embodiments 1-13, wherein the anti-Claudin-1 antibody comprises a VH comprising the amino acid sequence set forth in SEQ ID NO: 3; and a VL comprising the amino acid sequence set forth in SEQ ID NO: 4.

Embodiment 15. The method of any one of embodiments 1-14, wherein the anti-Claudin-1 antibody comprises a VH comprising the amino acid sequence set forth in SEQ ID NO: 13; and a VL comprising the amino acid sequence set forth in SEQ ID NO: 14.

Embodiment 16. The method of any one of embodiments 1-15, wherein the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

Embodiment 17. The method of any one of embodiments 1-16, wherein the anti-Claudin-1 antibody comprises a complementarity determining region (CDR) L1 comprising the amino acid sequence set forth in SEQ ID NO: 8, a CDR L2 comprising the amino acid sequence GA, and a CDR L3 comprising the amino acid sequence set forth in SEQ ID NO: 10

Embodiment 18. The method of embodiment 1, further comprising a therapeutically effective amount of sorafenib to the subject.

1. A method of treating hepatocellular carcinoma (HCC) in a human subject, comprising administering a therapeutically effective amount of an anti-Claudin-1 antibody or an antigen binding fragment thereof comprising:
a heavy chain complementarity determining region 1 (CDRH1) comprising the amino acid sequence of SEQ ID NO: 5;
a heavy chain complementarity determining region 2 (CDRH2) comprising the amino acid sequence of SEQ ID NO: 6;
a heavy chain complementarity determining region 3 (CDRH3) comprising the amino acid sequence of SEQ ID NO: 7;
a light chain complementarity determining region 1 (CDRL1) comprising the amino acid sequence of SEQ ID NO: 8;
a light chain complementarity determining region 2 (CDRL2) comprising the amino acid sequence of GA; and
a light chain complementarity determining region 3 (CDRL3) comprising the amino acid sequence of SEQ ID NO: 10,
wherein the HCC is resistant to sorafenib or a PD-1 antagonist.

2. The method of claim 1, wherein the anti-Claudin-1 antibody or antigen binding fragment thereof comprises a heavy chain variable region comprising an amino acid sequence of SEQ ID NO: 3 and a light chain variable region comprising an amino acid sequence of SEQ ID NO: 4.

3. The method of claim 1, wherein the anti-Claudin-1 antibody or antigen binding fragment thereof comprises a heavy chain variable region comprising an amino acid sequence of SEQ ID NO: 13 and a light chain variable region comprising an amino acid sequence of SEQ ID NO: 14.

4. A method for identifying a human subject having HCC that is suitable for therapy with an anti-Claudin-1 antibody or antigen binding fragment thereof comprising the steps of:
a) obtaining a biological sample a human subject having HCC;
b) detecting the expression of Claudin-1;
c) comparing the detected expression level of Claudin-1 with a control level of expression; and
d) identifying the human subject as a responder when the detected expression level of Claudin-1 is greater than the control level of expression;
wherein the anti-Claudin-1 antibody or antigen binding fragment thereof
a CDRH1 comprising the amino acid sequence of SEQ ID NO: 5;
a CDRH2 comprising the amino acid sequence of SEQ ID NO: 6;
a CDRH3 comprising the amino acid sequence of SEQ ID NO: 7;
a CDRL1 comprising the amino acid sequence of SEQ ID NO: 8;
a CDRL2 comprising the amino acid sequence of GA; and
a CDRL3 comprising the amino acid sequence of SEQ ID NO: 10.

5. The method of claim 4, comprising:
e) administering an anti-Claudin-1 antibody or antigen binding fragment thereof in an amount sufficient to alleviate a symptom of HCC when the human subject is identified as a responder.

6. A method for treating a human subject having HCC comprising the steps of:
a) obtaining a biological sample from a human subject having HCC;
b) detecting the expression levels of Claudin-1;
c) comparing the detected expression level of Claudin-1 with a control level of expression;
d) identifying the human subject as a responder when the detected expression level of Claudin-1 is greater than the control level of expression; and
e) administering an anti-Claudin-1 antibody or antigen binding fragment thereof in an amount sufficient to alleviate a symptom of HCC when the human subject is identified as a responder;
wherein the anti-Claudin-1 antibody or antigen binding fragment thereof
a CDRH1 comprising the amino acid sequence of SEQ ID NO: 5;
a CDRH2 comprising the amino acid sequence of SEQ ID NO: 6;
a CDRH3 comprising the amino acid sequence of SEQ ID NO: 7;
a CDRL1 comprising the amino acid sequence of SEQ ID NO: 8;
a CDRL2 comprising the amino acid sequence of GA; and
a CDRL3 comprising the amino acid sequence of SEQ ID NO: 10.

7. The method of claim 6, wherein the control level of expression is determined from a normal tissue sample, and wherein the normal tissue sample is adjacent to the biological sample from the human subject having HCC.

8. The method of claim 6, wherein the HCC is resistant to sorafenib or a PD-1 antagonist.

9. (canceled)

10. The method of claim 6, wherein the anti-Claudin-1 antibody or antigen binding fragment thereof comprises a heavy chain variable region comprising an amino acid sequence of SEQ ID NO: 3 and a light chain variable region comprising an amino acid sequence of SEQ ID NO: 4.

11. The method of claim 6, wherein the anti-Claudin-1 antibody or antigen binding fragment thereof comprises a heavy chain variable region comprising an amino acid sequence of SEQ ID NO: 13 and a light chain variable region comprising an amino acid sequence of SEQ ID NO: 14.

12. The method of claim 1, wherein the anti-Claudin-1 antibody is a humanized antibody.

13. The method of claim 1, wherein the human subject was previously treated with sorafenib.

14. The method of claim 1, wherein the human subject is simultaneously or sequentially administered with a therapeutically effective amount of sorafenib.

15. The method of claim 1, wherein the human subject was previously treated with a PD-1 antagonist.

16. The method of claim 1, wherein the human subject is simultaneously or sequentially administered with a therapeutically effective amount of PD-1 antagonist.

17. The method of claim 1, wherein the PD-1 antagonist is nivolumab, pembrolizumab, cemiplimab, dostarlimab or a combination thereof.

18. The method of claim 17, wherein the PD-1 antagonist is nivolumab.
